(19) United States
(12) Patent Application Publication
(10) Pub. No.: US 2025/0053867 A1
(43) Pub. Date: Feb. 13, 2025

(54) LEARNING APPARATUS, RECOMMENDER APPARATUS, METHODS AND PROGRAMS FOR THE SAME

(71) Applicant: NIPPON TELEGRAPH AND TELEPHONE CORPORATION, Tokyo (JP)

(72) Inventors: Naoki SAIJO, Tokyo (JP); Hiroshi UEDA, Tokyo (JP)

(73) Assignee: NIPPON TELEGRAPH AND TELEPHONE CORPORATION, Tokyo (JP)

(21) Appl. No.: 18/717,997

(22) PCT Filed: May 25, 2022
(86) PCT No.: PCT/JP2022/021363

§ 371 Date: Jun. 11, 2024

(30) Foreign Application Priority Data
Dec. 13, 2021 (WO) PCT/JP2021/045766

(51) Int. Cl. G06N 20/00

(52) U.S. Cl. CPC G06N 20/00

(57) ABSTRACT

From information for specifying a piece of music, biological information on a user before listening to the piece of music, and information regarding improvement in exercise capacity of the user, for a plurality of pieces of music, learning is performed of an estimation model that uses biological information as an input and obtains information on a piece of music estimated to have the largest effect of improving exercise capacity of the user in a state of the input biological information or information on a predetermined number of pieces of music in order from the piece of music estimated to have the largest effect, and by using the estimation model obtained by learning, on the basis of biological information on a user who is a target of recommendation of a piece of music, information on a piece of music estimated to have the largest effect of improving exercise capacity when the user is in a state of the biological information, or information on a predetermined number of pieces of music in order from the piece of music estimated to have the largest effect is obtained as a recommendation result.

### TECHNICAL FIELD

The present invention relates to a technique for obtaining recommendation information regarding a piece of music to be presented for temporarily enhancing exercise capacity of a user.

### BACKGROUND ART

Studies have been conventionally conducted on an influence of listening to music on activity of a body such as an autonomic nerve, and a sympathetic nerve among autonomic nerves. For example, Non Patent Literature 1 describes that heartbeat changes by listening to music. In addition, for example, Non Patent Literature 2 describes that listening to relaxing music or the like causes secretion of oxytocin in a brain and relaxation can be made.

### CITATION LIST

### Non Patent Literature

Non Patent Literature 1: Bernardi, L., Porta, C., & Sleight, P., “Cardiovascular, cerebrovascular, and respiratory changes induced by different types of music in musicians and non-musicians: The importance of silence”, Heart, 2006, 92 (4), 445-452.
Non Patent Literature 2: Ooishi, Y., Mukai, H., Watanabe, K., Kawato, S., & Kashino, M., “Increase in salivary oxytocin and decrease in salivary cortisol after listening to relaxing slow-tempo and exciting fast-tempo music”, PLOS ONE, 2017, 12 (12), 1-16.

### SUMMARY OF INVENTION

### Technical Problem

Conventional studies such as Non Patent Literature 1 and Non Patent Literature 2 are examinations on an influence of listening to music on activity of internal organs and nerves, and there is no known examination result on an influence of listening to music on exercise capacity. An object of the present invention is to provide a technique for obtaining recommendation information regarding a piece of music to be presented for temporarily enhancing exercise capacity of a user in consideration of the influence of listening to music on exercise capacity.

### Solution to Problem

To solve the above problem, according to one aspect of the present invention, in a learning apparatus, training data includes a learning information set for each of a plurality of pieces of music, and the learning information set for each piece of music is a set of: piece-of-music information that is information for specifying the piece of music; biological information acquired before listening to the piece of music for each of learning target users who are users targeted for learning; and an index value regarding improvement in exercise capacity due to listening to the piece of music for each of the learning target users, and the learning apparatus includes a learning unit that uses input training data and performs learning of an estimation model that uses biological information as an input and obtains information on a piece of music estimated to have the largest effect of improving exercise capacity of a user in a state of the input biological information.

To solve the above problem, according to another aspect of the present invention, in a learning apparatus, training data includes a learning information set for each of a plurality of pieces of music, and the learning information set for each piece of music is a set of: piece-of-music information that is information for specifying the piece of music; biological information acquired before listening to the piece of music for each of learning target users who are users targeted for learning; and an index value regarding improvement in exercise capacity due to listening to the piece of music for each of the learning target users, and the learning apparatus includes a learning unit that uses input training data and performs learning of an estimation model that uses biological information as an input and obtains information on a predetermined number of pieces of music in order from a piece of music estimated to have the largest effect of improving exercise capacity of a user in a state of the input biological information.

To solve the above problem, according to another aspect of the present invention, in a recommender apparatus, an estimation model that uses biological information as an input and obtains piece-of-music information on a piece of music estimated to have the largest effect of improving exercise capacity of a user in a state of the input biological information is stored in advance, and the recommender apparatus includes a piece-of-music recommendation information generation unit that uses the estimation model, uses biological information on a recommendation target user who is a user as a target of recommendation of a piece of music as an input, and obtains information on a piece of music estimated to have the largest effect of improving exercise capacity of the recommendation target user in a state of the biological information.

To solve the above problem, according to another aspect of the present invention, in a recommender apparatus, an estimation model that uses biological information as an input and obtains piece-of-music information on a predetermined number of pieces of music in order from a piece of music estimated to have the largest effect of improving exercise capacity of a user in a state of the input biological information is stored in advance, and the recommender apparatus includes a piece-of-music recommendation information generation unit that uses the estimation model, uses biological information on a recommendation target user who is a user as a target of recommendation of a piece of music as an input, and obtains information on a predetermined number of pieces of music in order from a piece of music estimated to have the largest effect of improving exercise capacity of the recommendation target user in a state of the biological information.

### Advantageous Effects of Invention

According to the present invention, it is possible to obtain recommendation information regarding a piece of music to be presented for temporarily enhancing exercise capacity of a user.

### BRIEF DESCRIPTION OF DRAWINGS

FIG. 1 is a block diagram illustrating an example of a recommender system including a learning apparatus and a recommender apparatus.

FIG. 2 is a diagram illustrating an example of a flow of processing performed by the learning apparatus.

FIG. 3 is a diagram illustrating an example of a flow of processing performed by the recommender apparatus.

FIG. 4 is a diagram illustrating an example configuration of a computer that functions as at least one of the learning apparatus or the recommender apparatus.

### DESCRIPTION OF EMBODIMENTS

### Background of Invention

It is known that sympathetic nerves are enhanced during exercise, and in consideration of results of the above-described prior studies that listening to music affects sympathetic nerves, a hypothesis holds that listening to music may lead to enhancement of exercise capacity. Thus, the inventor investigated the influence of listening to music on exercise capacity. Specifically, an experiment was conducted in which vertical jump was used as a specific example of exercise, a plurality of subjects was set as targets, for each of a plurality of pieces of music, the first jump was made and a height was measured before listening to a piece of music, the second jump was made and a height was measured after listening to the piece of music, and presence or absence of an improvement in the height of the jump and the degree of the improvement were confirmed. As a result of the experiment, it was confirmed that there was an improvement in the height of the jump due to listening to music. That is, it was confirmed that exercise capacity of a subject was temporarily enhanced by allowing the subject to listen to music. However, it was also confirmed that the improvement in the height of the jump depends on the subject and the piece of music.

Although a scientific mechanism from listening to music to improvement in exercise capacity has not yet been clarified at the present time, from the conventional study and the experimental results confirmed this time, if a set of: information of a piece of music; biological information on a user before listening to the piece of music; and information regarding improvement in exercise capacity of the user due to listening to the piece of music, is acquired in advance for a plurality of pieces of music, it should be possible to recommend what kind of piece of music needs to be presented for listening when it is desired to temporarily improve the exercise capacity of the user, on the basis of the biological information on the user, by using the information acquired in advance. Thus, the present invention performs learning of an estimation model for recommending a piece of music from a plurality of sets acquired for a plurality of pieces of music, each set comprising: information on a piece of music; biological information on a user before listening to the piece of music; and information regarding improvement in exercise capacity of the user due to listening to the piece of music, and obtains recommendation information regarding a piece of music that is expected to temporarily enhance exercise capacity of a user who is a target of recommendation of the piece of music, on the basis of the biological information on the user who is the target of recommendation of the piece of music, by using the estimation model obtained by the learning.

### First Embodiment

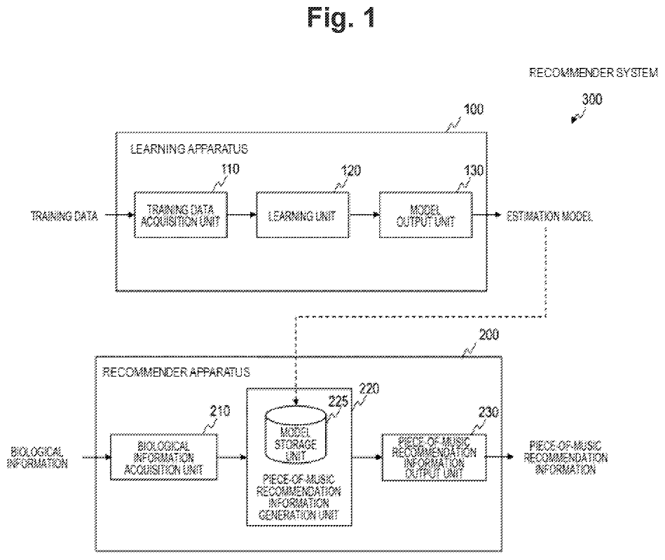

A recommender system according to a first embodiment obtains, as a recommendation result, information on a piece of music to be presented for temporarily enhancing exercise capacity of a user. As illustrated in FIG. 1, a recommender system 300 of the first embodiment includes a learning apparatus 100 and a recommender apparatus 200.

The learning apparatus and the recommender apparatus are special devices configured by loading a special program into a known or dedicated computer including, for example, a central processing unit (CPU) and a main memory (random access memory (RAM)). The learning apparatus and the recommender apparatus perform pieces of processing under control of the central processing unit, for example. Data input to the learning apparatus and the recommender apparatus and data obtained in the pieces of processing are stored in the main memory, for example, and the data stored in the main memory is read into the central processing unit and is used for other processing as necessary. In the learning apparatus and the recommender apparatus, at least a part of each of processing units may be configured by hardware such as an integrated circuit. Each of storage units included in the learning apparatus and the recommender apparatus can be configured by, for example, the main memory such as a random access memory (RAM), or middleware such as a relational database or a key-value store. However, each storage unit is not necessarily provided inside the learning apparatus and the recommender apparatus, and may be configured by an auxiliary recording device including a non-transitory recording medium such as a hard disk or an optical disk, and may be provided outside the learning apparatus and the recommender apparatus. The same applies to a learning apparatus and a recommender apparatus of a second embodiment described later.

First, the learning apparatus 100 will be described.

### [Learning Apparatus 100]

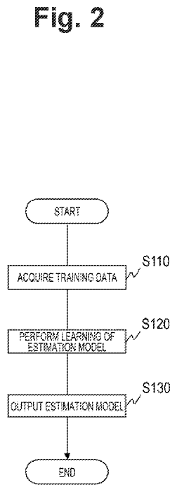

Training data including: information for specifying a piece of music; biological information on a user before listening to the piece of music; and information regarding improvement in exercise capacity of the user due to listening to the piece of music, for a plurality of pieces of music is input to the learning apparatus 100. The learning apparatus 100 uses input training data and performs learning of an estimation model that uses biological information as an input and obtains, as a recommendation result, information on a piece of music estimated to have the largest effect of improving exercise capacity of the user in a state of the biological information, and outputs a learned estimation model. As illustrated in FIG. 1, the learning apparatus 100 includes a training data acquisition unit 110, a learning unit 120, and a model output unit 130. The learning apparatus 100 performs processing of step S110, step S120, and step S130 illustrated in FIG. 2.

### [Training Data Acquisition Unit 110]

The training data acquisition unit 110 at least functions as an interface for reading, into the learning apparatus 100, training data acquired by a device different from the learning apparatus 100 and/or training data stored in a device different from the learning apparatus 100. The training data input to the learning apparatus 100 is input to the training data acquisition unit 110. The training data acquisition unit 110 outputs the input training data to the learning unit 120 (step S110).

The training data includes a learning information set for each of the plurality of pieces of music. The learning information set for each piece of music is a set of: piece-of-music information; biological information acquired before listening to the piece of music for each of users targeted for learning (hereinafter, referred to as “learning target users”); and an index value regarding improvement in exercise capacity due to listening to the piece of music for each learning target user. For example, when each of indexes 1, . . . , and J of the pieces of music is j and each of indexes 1, . . . , and K of the users at the time of learning is k, a learning information set S(j) for each of pieces of music M(j) is a set S(j)={A(j), B(j, 1), . . . , B(j, K), C(j, 1), . . . , C(j, K)} of: piece-of-music information A(j) of a piece of music M(j); biological information B(j, k) before listening to the piece of music M(j) for each of users U(k); and an index value C(j, k) regarding improvement in exercise capacity due to listening to the piece of music M(j) for each user U(k), and the training data includes J learning information sets S(1), . . . , and S(J).

The piece-of-music information A(j) is information for specifying one piece of music M(j) among the plurality of pieces of music M(1), . . . , and M(J), and is, for example, a title of the piece of music. The biological information B(j, k) is biological information on the user U(k) acquired before the user U(k) listens to the piece of music M(j), and is, for example, biological information such as an electrocardiogram, a heart rate, respiration, mental sweating, and a pupil diameter acquired by a sensor attached to the user U(k) before a first predetermined time before the user U(k) starts listening to the piece of music M(j). The index value C(j, k) regarding improvement in exercise capacity is an index value regarding improvement in exercise capacity due to listening to the piece of music M(j), which is obtained from a result of predetermined exercise performed before a second predetermined time before the user U(k) starts listening to the piece of music M(j) and a result of the predetermined exercise performed after a third predetermined time after the user U(k) finishes listening to the pieces of music M(j), and is, for example, a value representing an amount or degree of improvement in exercise capacity due to listening to the piece of music M(j). For example, when the predetermined exercise is vertical jump, and a height of the vertical jump performed before the second predetermined time before the user U(k) starts listening to the piece of music M(j) is H₁(k), and a height of the vertical jump performed after the third predetermined time after the user U(k) finishes listening to the piece of music M(j) is H₂(k), it is sufficient that, for example, a value H₂(k)−H₁(k) obtained by subtracting H₁(k) from H₂(k), a value H₂(k)/H₁(k) obtained by dividing H₂(k) by H₁(k), a value (H₂(k)−H₁(k))/H₁(k) obtained by dividing the value H₂(k)−H₁(k) obtained by subtracting H₁(k) from H₂(k) by H₁(k), or a value (H₂(k)−H₁(k))/H₂(k) obtained by dividing the value H₂(k)−H₁(k) obtained by subtracting H₁(k) from H₂(k) by H₂(k) is set as the index value C(j, k) regarding improvement in exercise capacity. It is sufficient that the first predetermined time, the second predetermined time, and the third predetermined time are determined in advance by experiments or the like. Note that the first predetermined time is preferably longer than the second predetermined time so that the biological information B(j, k) is not affected by exercise.

Note that the training data acquisition unit 110 may obtain another piece of biological information B′(j, k) from the input biological information B(j, k), and output the biological information B′(j, k) instead of the biological information B(j, k). For example, the training data acquisition unit 110 may obtain the biological information B′(j, k) representing a psychological state of the user U(k) from the biological information B(j, k) acquired from the user U(k), and output the biological information B′(j, k) representing the psychological state of the user U(k) instead of the biological information B(j, k) acquired from the user U(k). The biological information B′(j, k) is, for example, an index value of tension. For example, in a case where the input biological information B(j, k) is a waveform of the electrocardiogram and an amount of sweating of mental sweating, the training data acquisition unit 110 may obtain the index value of tension as the biological information B′(j, k) from the waveform of the electrocardiogram and the amount of sweating of mental sweating, and output the biological information B′(j, k). That is, the training data acquisition unit 110 may output a part of the input learning data converted, to the learning unit 120 as training data.

### [Learning Unit 120]

The training data output by the training data acquisition unit 110 is input to the learning unit 120. The learning unit 120 uses input training data and performs learning of an estimation model that uses biological information as an input and obtains information on a piece of music estimated to have the largest effect of improving exercise capacity of the user in a state of the biological information (step S120), and outputs a learned estimation model to the model output unit 130. It is sufficient that a known learning technique is used for learning of the estimation model, and an amount of training data is a sufficient amount for performing learning of the estimation model.

The learning unit 120 may perform learning of the estimation model by using the biological information and index value regarding improvement in exercise capacity on all users at the time of learning, may perform learning of the estimation model by using the biological information and index value regarding improvement in exercise capacity on a user satisfying a predetermined condition among the users at the time of learning, or may perform learning of the estimation model by using the biological information and index value regarding improvement in exercise capacity on one specific user among the users at the time of learning. In a case where the learning unit 120 performs learning by using the biological information and index value regarding improvement in exercise capacity on all the users, it is possible to obtain an estimation model having a low degree of dependence on the user. In a case where the learning unit 120 performs learning by using the biological information and index value regarding improvement in exercise capacity on a user satisfying the predetermined condition, it is possible to obtain an estimation model having a high degree of dependence on the user satisfying the condition. In a case where the learning unit 120 performs learning by using the biological information and index value regarding improvement in exercise capacity on one specific user, it is possible to obtain an estimation model specialized for the user.

### [Model Output Unit 130]

The learned estimation model output by the learning unit 120 is input to the model output unit 130. The model output unit 130 outputs the input learned estimation model to the recommender apparatus 200 as an output of the learning apparatus 100 (step S130).

Next, the recommender apparatus 200 will be described.

### [Recommender Apparatus 200]

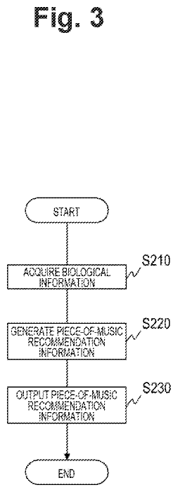

Biological information on a user as a target of recommendation of a piece of music (hereinafter, referred to as a “recommendation target user”) is input to the recommender apparatus 200. The recommender apparatus 200 uses the learned estimation model, uses the biological information on the recommendation target user as an input, and obtains, as a recommendation result, piece-of-music information on a piece of music estimated to have the largest effect of improving exercise capacity when the recommendation target user is in a state of the biological information, and outputs the piece-of-music information. As illustrated in FIG. 1, the recommender apparatus 200 includes a biological information acquisition unit 210, a piece-of-music recommendation information generation unit 220, and a piece-of-music recommendation information output unit 230. The recommender apparatus 200 performs processing of step S210, step S220, and step S230 illustrated in FIG. 3.

### [Biological Information Acquisition Unit 210]

The biological information on the recommendation target user input to the recommender apparatus 200 is input to the biological information acquisition unit 210. The biological information acquisition unit 210 outputs the input biological information to the piece-of-music recommendation information generation unit 220 (step S210).

The biological information on the recommendation target user is, for example, an electrocardiogram, a heart rate, respiration, mental sweating, and a pupil diameter acquired by a sensor attached to the recommendation target user. Note that, in a case where the training data acquisition unit 110 of the corresponding learning apparatus 100 obtains and outputs another piece of biological information from the input biological information, the biological information acquisition unit 210 also obtains and outputs another piece of biological information from the input biological information, similarly to the training data acquisition unit 110. An example of another piece of biological information is an index value of tension. For example, in a case where the training data acquisition unit 110 of the corresponding learning apparatus 100 obtains and outputs the index value of tension as the biological information from the waveform of the electrocardiogram and the amount of sweating of mental sweating that are input, the biological information acquisition unit 210 also obtains and outputs the index value of tension as the biological information from the waveform of the electrocardiogram and the amount of sweating of mental sweating that are input. That is, the biological information acquisition unit 210 may output the input biological information converted, to the piece-of-music recommendation information generation unit 220 as the biological information.

### Piece-of-Music Recommendation Information Generation Unit 220

As illustrated in FIG. 1, the piece-of-music recommendation information generation unit 220 includes a model storage unit 225. The learned estimation model output from the corresponding learning apparatus 100 is stored in advance in the model storage unit 225. The learned estimation model stored in advance in the model storage unit 225 is an estimation model that uses biological information as an input and obtains piece-of-music information on a piece of music estimated to have the largest effect of improving exercise capacity of the user in a state of the biological information.

The biological information output from the biological information acquisition unit 210 is input to the piece-of-music recommendation information generation unit 220. The piece-of-music recommendation information generation unit 220 uses the learned estimation model stored in advance in the model storage unit 225, uses biological information on the recommendation target user as an input, and obtains, as piece-of-music recommendation information, piece-of-music information on a piece of music estimated to have the largest effect of improving exercise capacity of the recommendation target user in a state of the biological information (step S220), and outputs the obtained piece-of-music recommendation information to the piece-of-music recommendation information output unit 230.

The learned estimation model stored in advance in the model storage unit 225 may be obtained by any learning performed by the learning unit 120 of the learning apparatus 100. That is, the learned estimation model stored in advance in the model storage unit 225 may be an estimation model learned by using the biological information and index value regarding improvement in exercise capacity on all users at the time of learning, may be an estimation model learned by using the biological information and index value regarding improvement in exercise capacity on a user satisfying a predetermined condition among the users at the time of learning, or may be an estimation model learned by using the biological information and index value regarding improvement in exercise capacity on one specific user among the users at the time of learning. However, in a case where the estimation model is used learned by using the biological information and index value regarding improvement in exercise capacity on a user satisfying a predetermined condition among the users at the time of learning, it is preferable to use an estimation model learned by using the biological information and index value regarding improvement in exercise capacity on the user satisfying a condition satisfied by the recommendation target user. In addition, in a case where the estimation model is used learned by using the biological information and index value regarding improvement in exercise capacity on one specific user among the users at the time of learning, it is preferable to use an estimation model learned by using the biological information and index value regarding improvement in exercise capacity on the recommendation target user.

### Piece-of-Music Recommendation Information Output Unit 230

The piece-of-music recommendation information output by the piece-of-music recommendation information generation unit 220 is input to the piece-of-music recommendation information output unit 230. The piece-of-music recommendation information output unit 230 outputs the input music recommendation information as an output of the recommender apparatus 200 (step S230).

### Second Embodiment

A recommender system of a second embodiment obtains, as a recommendation result, a playlist of pieces of music to be presented for temporarily enhancing exercise capacity of a user. The playlist of pieces of music is information on a plurality of pieces of music. As illustrated in FIG. 1, a recommender system 300 of the second embodiment includes a learning apparatus 100 and a recommender apparatus 200.

First, the learning apparatus 100 will be described.

### [Learning Apparatus 100]

Training data including: information for specifying a piece of music; biological information on a user before listening to the piece of music; and information regarding improvement in exercise capacity of the user due to listening to the piece of music, for a plurality of pieces of music is input to the learning apparatus 100. The learning apparatus 100 uses input training data and performs learning of an estimation model that uses biological information as an input and obtains, as a recommendation result, a playlist of pieces of music estimated to have large effects of improving exercise capacity of the user in a state of the biological information, and outputs a learned estimation model. As illustrated in FIG. 1, the learning apparatus 100 includes a training data acquisition unit 110, a learning unit 120, and a model output unit 130. The learning apparatus 100 performs processing of step S110, step S120, and step S130 illustrated in FIG. 2.

### [Training Data Acquisition Unit 110]

The training data acquisition unit 110 at least functions as an interface for reading, into the learning apparatus 100, training data acquired by a device different from the learning apparatus 100 and/or training data stored in a device different from the learning apparatus 100. The training data input to the learning apparatus 100 is input to the training data acquisition unit 110. The training data acquisition unit 110 outputs the input training data to the learning unit 120 (step S110).

The training data includes a learning information set for each of the plurality of pieces of music. The learning information set for each piece of music is a set of: piece-of-music information; biological information acquired before listening to the piece of music for each of users targeted for learning (hereinafter, referred to as “learning target users”); and an index value regarding improvement in exercise capacity due to listening to the piece of music for each learning target user. For example, when each of indexes 1, . . . , and J of the pieces of music is j and each of indexes 1, . . . , and K of the users at the time of learning is k, a learning information set S(j) for each of pieces of music M(j) is a set S(j)={A(j), B(j, 1), . . . , B(j, K), C(j, 1), . . . , C(j, K)} of: piece-of-music information A(j) of a piece of music M(j); biological information B(j, k) before listening to the piece of music M(j) for each of users U(k); and an index value C(j, k) regarding improvement in exercise capacity due to listening to the piece of music M(j) for each user U(k), and the training data includes J learning information sets S(1), . . . , and S(J).

The piece-of-music information A(j) is information for specifying one piece of music M(j) among the plurality of pieces of music M(1), . . . , and M(J), and is, for example, a title of the piece of music. The biological information B(j, k) is biological information on the user U(k) acquired before the user U(k) listens to the piece of music M(j), and is, for example, biological information such as an electrocardiogram, a heart rate, respiration, mental sweating, and a pupil diameter acquired by a sensor attached to the user U(k) before a first predetermined time before the user U(k) starts listening to the piece of music M(j). The index value C(j, k) regarding improvement in exercise capacity is an index value regarding improvement in exercise capacity due to listening to the piece of music M(j), which is obtained from a result of predetermined exercise performed before a second predetermined time before the user U(k) starts listening to the piece of music M(j) and a result of the predetermined exercise performed after a third predetermined time after the user U(k) finishes listening to the pieces of music M(j), and is, for example, a value representing an amount or degree of improvement in exercise capacity due to listening to the piece of music M(j). For example, when the predetermined exercise is vertical jump, and a height of the vertical jump performed before the second predetermined time before the user U(k) starts listening to the piece of music M(j) is H₁(k), and a height of the vertical jump performed after the third predetermined time after the user U(k) finishes listening to the piece of music M(j) is H₂(k), it is sufficient that, for example, a value H₂(k)−H₁(k) obtained by subtracting H (k) from H₂(k), a value H₂(k)/H₁(k) obtained by dividing H₂(k) by H₁(k), a value (H₂(k)−H₁(k))/H₁(k) obtained by dividing the value H₂(k)−H₁(k) obtained by subtracting H₁(k) from H₂(k) by H₁(k), or a value (H₂(k)−H₁(k))/H₂(k) obtained by dividing the value H₂(k)−H₁(k) obtained by subtracting H₁(k) from H₂(k) by H₂(k) is set as the index value C(j, k) regarding improvement in exercise capacity. It is sufficient that the first predetermined time, the second predetermined time, and the third predetermined time are determined in advance by experiments or the like. Note that the first predetermined time is preferably longer than the second predetermined time so that the biological information B(j, k) is not affected by exercise.

Note that the training data acquisition unit 110 may obtain another piece of biological information B′(j, k) from the input biological information B(j, k), and output the biological information B′(j, k) instead of the biological information B(j, k). For example, the training data acquisition unit 110 may obtain the biological information B′(j, k) representing a psychological state of the user U(k) from the biological information B(j, k) acquired from the user U(k), and output the biological information B′(j, k) representing the psychological state of the user U(k) instead of the biological information B(j, k) acquired from the user U(k). The biological information B′(j, k) is, for example, an index value of tension. For example, in a case where the input biological information B(j, k) is a waveform of the electrocardiogram and an amount of sweating of mental sweating, the training data acquisition unit 110 may obtain the index value of tension as the biological information B′(j, k) from the waveform of the electrocardiogram and the amount of sweating of mental sweating, and output the biological information B′(j, k). That is, the training data acquisition unit 110 may output a part of the input learning data converted, to the learning unit 120 as training data.

### [Learning Unit 120]

The training data output by the training data acquisition unit 110 is input to the learning unit 120. The learning unit 120 uses input training data and performs learning of an estimation model that uses biological information as an input and obtains information on a plurality of pieces of music estimated to have large effects of improving exercise capacity of the user in a state of the biological information, and outputs a learned estimation model to the model output unit 130. Specifically, the learning unit 120 uses input training data and performs learning of an estimation model that uses biological information as an input and obtains piece-of-music information on a predetermined number of pieces of music in order from a piece of music estimated to have the largest effect of improving exercise capacity of the user in a state of the biological information (step S120), and outputs a learned estimation model to the model output unit 130. It is sufficient that a known learning technique is used for learning of the estimation model, and an amount of training data is a sufficient amount for performing learning of the estimation model.

The learning unit 120 may perform learning of the estimation model by using the biological information and index value regarding improvement in exercise capacity on all users at the time of learning, may perform learning of the estimation model by using the biological information and index value regarding improvement in exercise capacity on a user satisfying a predetermined condition among the users at the time of learning, or may perform learning of the estimation model by using the biological information and index value regarding improvement in exercise capacity on one specific user among the users at the time of learning. In a case where the learning unit 120 performs learning by using the biological information and index value regarding improvement in exercise capacity on all the users, it is possible to obtain an estimation model having a low degree of dependence on the user. In a case where the learning unit 120 performs learning by using the biological information and index value regarding improvement in exercise capacity on a user satisfying the predetermined condition, it is possible to obtain an estimation model having a high degree of dependence on the user satisfying the condition. In a case where the learning unit 120 performs learning by using the biological information and index value regarding improvement in exercise capacity on one specific user, it is possible to obtain an estimation model specialized for the user.

### [Model Output Unit 130]

The learned estimation model output by the learning unit 120 is input to the model output unit 130. The model output unit 130 outputs the input learned estimation model to the recommender apparatus 200 as an output of the learning apparatus 100 (step S130).

Next, the recommender apparatus 200 will be described.

### [Recommender Apparatus 200]

Biological information on a user as a target of recommendation of a piece of music (hereinafter, referred to as a “recommendation target user”) is input to the recommender apparatus 200. The recommender apparatus 200 uses the learned estimation model, uses the biological information on the recommendation target user as an input, and obtains, as a recommendation result, a playlist of pieces of music estimated to have large effects of improving exercise capacity when the recommendation target user is in a state of the biological information, and outputs the playlist. As illustrated in FIG. 1, the recommender apparatus 200 includes a biological information acquisition unit 210, a piece-of-music recommendation information generation unit 220, and a piece-of-music recommendation information output unit 230. The recommender apparatus 200 performs processing of step S210, step S220, and step S230 illustrated in FIG. 3.

### [Biological Information Acquisition Unit 210]

The biological information on the recommendation target user input to the recommender apparatus 200 is input to the biological information acquisition unit 210. The biological information acquisition unit 210 outputs the input biological information to the piece-of-music recommendation information generation unit 220 (step S210).

The biological information on the recommendation target user is, for example, an electrocardiogram, a heart rate, respiration, mental sweating, and a pupil diameter acquired by a sensor attached to the recommendation target user. Note that, in a case where the training data acquisition unit 110 of the corresponding learning apparatus 100 obtains and outputs another piece of biological information from the input biological information, the biological information acquisition unit 210 also obtains and outputs another piece of biological information from the input biological information, similarly to the training data acquisition unit 110. An example of another piece of biological information is an index value of tension. For example, in a case where the training data acquisition unit 110 of the corresponding learning apparatus 100 obtains and outputs the index value of tension as the biological information from the waveform of the electrocardiogram and the amount of sweating of mental sweating that are input, the biological information acquisition unit 210 also obtains and outputs the index value of tension as the biological information from the waveform of the electrocardiogram and the amount of sweating of mental sweating that are input. That is, the biological information acquisition unit 210 may output the input biological information converted, to the piece-of-music recommendation information generation unit 220 as the biological information.

### Piece-of-Music Recommendation Information Generation Unit 220

As illustrated in FIG. 1, the piece-of-music recommendation information generation unit 220 includes a model storage unit 225. The learned estimation model output from the corresponding learning apparatus 100 is stored in advance in the model storage unit 225. The learned estimation model stored in advance in the model storage unit 225 is an estimation model that uses biological information as an input and obtains piece-of-music information on a predetermined number of pieces of music in order from a piece of music estimated to have the largest effect of improving exercise capacity of the user in a state of the biological information.

The biological information output from the biological information acquisition unit 210 is input to the piece-of-music recommendation information generation unit 220. The piece-of-music recommendation information generation unit 220 uses the learned estimation model stored in advance in the model storage unit 225, uses biological information on the recommendation target user as an input, and obtains, as piece-of-music recommendation information, piece-of-music information on a predetermined number of pieces of music in order from a piece of music estimated to have the largest effect of improving exercise capacity of the recommendation target user in a state of the biological information (step S220), and outputs the obtained piece-of-music recommendation information to the piece-of-music recommendation information output unit 230.

The learned estimation model stored in advance in the model storage unit 225 may be obtained by any learning performed by the learning unit 120 of the learning apparatus 100. That is, the learned estimation model stored in advance in the model storage unit 225 may be an estimation model learned by using the biological information and index value regarding improvement in exercise capacity on all users at the time of learning, may be an estimation model learned by using the biological information and index value regarding improvement in exercise capacity on a user satisfying a predetermined condition among the users at the time of learning, or may be an estimation model learned by using the biological information and index value regarding improvement in exercise capacity on one specific user among the users at the time of learning. However, in a case where the estimation model is used learned by using the biological information and index value regarding improvement in exercise capacity on a user satisfying a predetermined condition among the users at the time of learning, it is preferable to use an estimation model learned by using the biological information and index value regarding improvement in exercise capacity on the user satisfying a condition satisfied by the recommendation target user. In addition, in a case where the estimation model is used learned by using the biological information and index value regarding improvement in exercise capacity on one specific user among the users at the time of learning, it is preferable to use an estimation model learned by using the biological information and index value regarding improvement in exercise capacity on the recommendation target user.

### Piece-of-Music Recommendation Information Output Unit 230

The piece-of-music recommendation information output by the piece-of-music recommendation information generation unit 220 is input to the piece-of-music recommendation information output unit 230. The piece-of-music recommendation information output unit 230 outputs the input music recommendation information as an output of the recommender apparatus 200 (step S230).

### Other Examples, Etc.

In the first embodiment and the second embodiment, the example has been described in which the predetermined exercise is the vertical jump, but the predetermined exercise is naturally not limited to the vertical jump, and may be any exercise. However, in view of the background of the invention described above, the predetermined exercise is desirably instantaneous exercise such as ball throwing, throwing, short distance running, and long jump. In other words, the recommender system 300 of the first embodiment can be expected to be particularly effective in a case where instantaneous exercise is a target. That is, as the index value regarding improvement in exercise capacity included in the learning information set handled by the learning apparatus 100, an index value regarding an exercise result of the instantaneous exercise may be used, and more specifically, a value representing the amount or degree of improvement in the exercise result of the instantaneous exercise may be used. In this way, the learning apparatus 100 can perform learning of an estimation model that obtains information on a piece of music estimated to have the largest effect of improving the exercise result of instantaneous exercise of the user or information on a predetermined number of pieces of music in order from the piece of music estimated to have the largest effect, and the recommender apparatus 200 can obtain information on a piece of music estimated to have the largest effect of improving the exercise result of instantaneous exercise of the recommendation target user or information on a predetermined number of pieces of music in order from the piece of music estimated to have the largest effect.

### <Program and Recording Medium>

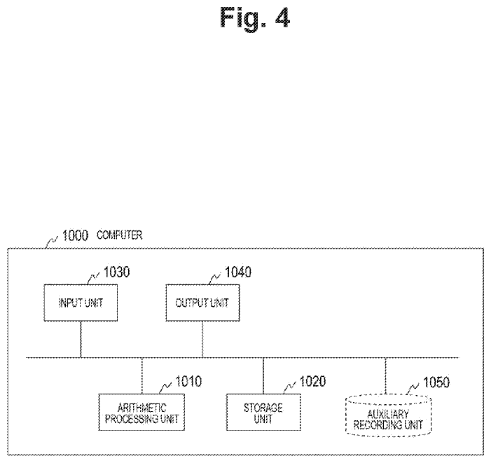

The processing of each unit of the learning apparatus and the recommender apparatus described above may be implemented by a computer, in which case, processing content of a function that each of the devices should have is described by a program. Then, by causing a storage unit 1020 of a computer 1000 illustrated in FIG. 4 to read this program and causing an arithmetic processing unit 1010, an input unit 1030, an output unit 1040, and the like to execute the program, various processing functions in each of the foregoing devices are implemented on the computer.

The program in which the processing content is described can be recorded in a computer-readable recording medium. The computer-readable recording medium is, for example, a non-transitory recording medium and is specifically a magnetic recording device, an optical disk, or the like.

In addition, the program is distributed by, for example, selling, transferring, or renting a portable recording medium such as a DVD and a CD-ROM in which the program is recorded. Further, the program may be stored in a storage device of a server computer, and the program may be distributed by transferring the program from the server computer to another computer via a network.

For example, the computer that performs such a program first temporarily stores the program recorded in a portable recording medium or the program transferred from the server computer in an auxiliary recording unit 1050 that is a non-transitory storage device of the computer. Then, at the time of performing processing, the computer reads the program stored in the auxiliary recording unit 1050 that is the non-transitory storage device of the computer into the storage unit 1020 and performs processing in accordance with the read program. In addition, as another embodiment of the program, the computer may directly read the program from the portable recording medium into the storage unit 1020 and perform processing in accordance with the program, and further, the computer may sequentially perform processing in accordance with a received program each time the program is transferred from the server computer to the computer. In addition, the above-described processing may be performed by a so-called application service provider (ASP) type service that implements a processing function only by a performance instruction and result acquisition without transferring the program from the server computer to the computer. Note that the program in the present embodiment includes information used for processing by an electronic computer and equivalent to the program (data or the like that is not a direct command to the computer but has property that defines processing performed by the computer).

In addition, although the present devices are each configured by executing a predetermined program on a computer in the present embodiments, at least part of the processing content may be implemented by hardware.

In addition, it is needless to say that modifications can be appropriately made without departing from the gist of the present invention.

1. A learning apparatus, wherein
training data includes a learning information set for each of a plurality of pieces of music, and
the learning information set for each piece of music is a set of: piece-of-music information that is information for specifying the piece of music; biological information acquired before listening to the piece of music for each of learning target users who are users targeted for learning; and an index value regarding improvement in exercise capacity due to listening to the piece of music for each of the learning target users,
the learning apparatus comprising processing circuitry configured to use input training data and perform learning of an estimation model that uses biological information as an input and obtains information on a piece of music estimated to have a largest effect of improving exercise capacity of a user in a state of the input biological information.

2. The learning apparatus according to claim 1, wherein
the estimation model obtains information on a predetermined number of pieces of music in order from the piece of music estimated to have the largest effect.

3. A recommender apparatus using the estimation model learned by the learning apparatus according to claim 1, wherein
the recommender apparatus comprises processing circuitry configured to use the estimation model, use biological information on a recommendation target user who is a user as a target of recommendation of a piece of music as an input, and obtain information on a piece of music estimated to have a largest effect of improving exercise capacity of the recommendation target user in a state of the biological information.

4. A recommender apparatus using the estimation model learned by the learning apparatus according to claim 2, wherein
the recommender apparatus comprises processing circuitry configured to use the estimation model, use biological information on a recommendation target user who is a user as a target of recommendation of a piece of music as an input, and obtain information on a predetermined number of pieces of music in order from a piece of music estimated to have a largest effect of improving exercise capacity of the recommendation target user in a state of the biological information.

5. A learning method, implemented by a learning apparatus that includes processing circuitry, wherein
training data includes a learning information set for each of a plurality of pieces of music, and
the learning information set for each piece of music is a set of: piece-of-music information that is information for specifying the piece of music; biological information acquired before listening to the piece of music for each of learning target users who are users targeted for learning; and an index value regarding improvement in exercise capacity due to listening to the piece of music for each of the learning target users,
the learning method comprising a learning step in which the processing circuitry uses input training data and performs learning of an estimation model that uses biological information as an input and obtains information on a piece of music estimated to have a largest effect of improving exercise capacity of a user in a state of the input biological information.

6. The learning method according to claim 5, wherein
the estimation model obtains information on a predetermined number of pieces of music in order from the piece of music estimated to have the largest effect.

7. A recommender method, implemented by a recommender apparatus that includes processing circuitry, wherein
an estimation model that uses biological information as an input and obtains piece-of-music information on a piece of music estimated to have a largest effect of improving exercise capacity of a user in a state of the input biological information is stored in advance,
the recommender method comprising a piece-of-music recommendation information generation step in which the processing circuitry uses the estimation model, uses biological information on a recommendation target user who is a user as a target of recommendation of a piece of music as an input, and obtains information on a piece of music estimated to have a largest effect of improving exercise capacity of the recommendation target user in a state of the biological information.

8. The recommender method according to claim 7, wherein
the estimation model obtains piece-of-music information on a predetermined number of pieces of music in order from the piece of music estimated to have the largest effect,
in the piece-of-music recommendation information generation step, the processing circuitry obtains information on a predetermined number of pieces of music in order from the piece of music estimated to have the largest effect of improving the exercise capacity of the recommendation target user in the state of the biological information.

9. A non-transitory computer-readable recording medium having recorded thereon a program for causing a computer to function as the learning apparatus according to claim 1.

10. A non-transitory computer-readable recording medium having recorded thereon a program causing a computer to function as the recommender apparatus according to claim 3.
